(19) United States
(12) Patent Application Publication
(10) Pub. No.: US 2009/0047656 A1
(43) Pub. Date: Feb. 19, 2009

(54) Molecular analysis of primary cells

(76) Inventors: Jonathan F. Baden, Bridgewater, NJ (US); Chang H. Choi, Piscataway, NJ (US); Dondapati Chowdary, Princeton Junction, NJ (US); Kathleen M. Curtin, Roselle Park, NJ (US); Tatiana Vener, Stirling, NJ (US); Haiying Wang, Bridgewater, NJ (US); Christine A. Burnett, Somerville, NJ (US); Abhijit Mazumder, Basking Ridge, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/717,835

(22) Filed: Mar. 13, 2007

Related U.S. Application Data
(60) Provisional application No. 60/781,882, filed on Mar. 13, 2006.
(60) Provisional application No. 60/781,901, filed on Mar. 13, 2006.

(51) Int. Cl. C12Q 1/70; C12Q 1/68; G01N 33/53; C12Q 1/02

(52) U.S. Cl. 435/5; 435/6; 435/29; 435/7.1; 435/7.21; 435/7.2

(57) ABSTRACT

The present invention provides a method of propagating cells of interest obtained from a biological specimen by a) enriching the cells under conditions that maintain sufficient cell viability; and b) propagating the cells under conditions effective to allow cell viability, proliferation and integrity.

### BACKGROUND OF INVENTION

Metastases are the leading cause of death in patients diagnosed with a primary tumor. Cancer metastasis occurs when cells shed from the primary tumor and disseminate to distant parts of the body though the peripheral blood stream or lymphatic drainage. The presence of CTCs in peripheral blood has been shown to be associated with decreased progression-free survival and decreased overall survival in patients treated for metastatic breast cancer. Although mechanical forces or an individual's immune response kills a number of these tumor cells entering the blood stream, it is known that a percentage of tumor cells survive and can be analyzed. The presence, enumeration and characterization of these rare epithelial cells in whole blood could provide valuable diagnostic and clinical information. Approximately 70-80% of all solid tumors originate from epithelial cells, which are not normally found in circulation. The comprehensive analyses of mRNA of circulating epithelial cells in the peripheral blood may provide valuable information on tumor load prognosis and treatment efficacy. For example the Her-2 receptor is over expressed in only 30% of breast cancer patients, which suggests that Herceptin would be an ineffective therapy for all patients. Thus, molecular profiling of CTCs should lead to improved characterization of CTCs and ultimately to development of more effective, personalized novel therapeutic strategies.

Early detection of cancer and its metastatic status are critical for the effective treatment of cancers leading to overall survival rate and improved quality of life. Metastases result from the spread of tumor cells shed from the primary tissue reaching different tissues through peripheral blood often referred as circulating tumor cells (CTCs). Presence of these CTCs in blood as detected by CellSearch™ technology has been shown to be associated with decreased survival rate thus serving as predictable “markers” for cancer progression (metastasis). These CTCs could potentially be used for pharmacogenomic studies (example, chemosensitivity). Additionally, molecular profiling studies can be carried out on the CTCs which should further lead us to better understanding of underlying mechanisms of metastatic potential/progression, prognosis and even therapeutic utility. The challenges are several fold: recovery of quality nucleic acids from CTCs; their availability in very limited quantity; sensitivity limitations of the existing assays; application/validation of existing marker sets for the CTCs. Furthermore, the molecular profiling always may not lead to accurate results due to the contamination of the captured CTCs with leukocytes whose expression profile may interfere with the results.

Adapting the CTCs to grow in vitro could result in propagating the cells to sufficient levels and alleviate the afore-mentioned challenges. The cells thus propagated could be used for various applications including assessing the clonality of different cell populations, discovery of signatures, development of assays using such signatures, fluorescent in situ hybridization (FISH) and immuno-histochemistry (IHC).

Early detection of cancer and its metastatic status are critical for the effective treatment of cancers leading to increased survival rate dramatically and improved quality of life. Metastases result from the spread of tumor cells shed from the primary tissue reaching different tissues through peripheral blood often referred as circulating tumor cells (CTCs). Presence of these CTCs in blood as detected by CellSearch™ technology has been shown to be associated with decreased survival rate thus serving as predictable “markers” for cancer progression (metastasis). These CTCs could potentially be used for pharmacogenomic studies (example, chemosensitivity).

Gene expression in cancer can be disrupted either through genetic alteration or epigenetic alteration, which alter the heritable state of gene expression. The main epigenetic modification of the human genome is methylation of cytosine residues within the context of the CpG dinucleotide. DNA methylation is interesting from a diagnostic viewpoint because it may be easily detected in cells released from neoplastic and pre-neoplastic lesions into serum, urine or sputum. And from a therapeutic viewpoint because epigenetically silenced genes may be reactivated by inhibitors of DNA methylation and/or histone deacetylase.

Recently, a study involving molecular characterization of the CTCs has been published that utilized expression profiling both by GeneChip® analysis and quantitative reverse transcription-PCR. The specimens used in this study had >100 CTCs which is much higher than what is typically seen in early screening (<10 CTCs). We pursue that Quantitative Multiplex Methylation Specific PCR (QMSP) technology to perform pre-amplification (nested PCR) to obtain enough of target DNA from small amount DNA captured CTCs (<5 cells).

### SUMMARY OF THE INVENTION

The present invention provides methods, apparatus and kits for sample processing of circulating tumor cells (CTC) within peripheral blood and assessing their gene expression profiles while providing support for the CellSearch™ platform for disease recurrence testing. The CellSearch™ Profile Kit is intended for the isolation of CTCs of epithelial origin in whole blood in conjunction with the CellSearch® AutoPrep System. The CellSearch™ Profile Kit contains a ferrofluid-based capture reagent, which consists of nano-particles with a magnetic core surrounded by a polymeric layer coated with antibodies targeting the Epithelial Cell Adhesion Molecule (EpCAM) antigen for capturing CTCs. The CellTracks™ AutoPrep System automates and standardizes processing by precisely dispensing reagents and timing magnetic incubation steps, offering scientists advanced tools to reproducibly and efficiently isolate CTCs for important research in a variety of carcinomas. The vast majority of leukocytes and other blood components are depleted from the enriched sample, thereby minimizing background. Further analysis is performed using established molecular biology techniques including RT-PCR and multiplex RT-PCR. The Molecular characterization assay is a molecular diagnostic assay that is intended for use following CTC enrichment. This assay incorporates both epithelial and tissue of origin markers to confirm circulating cells in a patient previously diagnosed and treated for breast cancer are in fact breast in origin.

### BRIEF DESCRIPTION OF THE DRAWINGS

FIG. 1 is a graph depicting RNA stability over time.

FIG. 2 is a graph depicting prostate-specific mRNA obtained from circulating tumor cells.

FIG. 3 is a graph depicting prostate-specific mRNA obtained from circulating tumor cells.

FIG. 4 depicts the results from A) 100 ng PBL DNA Spiking; or B) in 500 ng PBL DNA Spiking.

### DETAILED DESCRIPTION

A Biomarker is any indicia of an indicated Marker nucleic acid/protein. Nucleic acids can be any known in the art including, without limitation, nuclear, mitochondrial (homeoplasmy, heteroplasmy), viral, bacterial, fungal, mycoplasmal, etc. The indicia can be direct or indirect and measure over- or under-expression of the gene given the physiologic parameters and in comparison to an internal control, placebo, normal tissue or another carcinoma. Biomarkers include, without limitation, nucleic acids and proteins (both over and under-expression and direct and indirect). Using nucleic acids as Biomarkers can include any method known in the art including, without limitation, measuring DNA amplification, deletion, insertion, duplication, RNA, micro RNA (miRNA), loss of heterozygosity (LOH), single nucleotide polymorphisms (SNPs, Brookes (1999)), copy number polymorphisms (CNPs) either directly or upon genome amplification, microsatellite DNA, epigenetic changes such as DNA hypo- or hyper-methylation and FISH. Using proteins as Biomarkers includes any method known in the art including, without limitation, measuring amount, activity, modifications such as glycosylation, phosphorylation, ADP-ribosylation, ubiquitination, etc., or immunohistochemistry (IHC) and turnover. Other Biomarkers include imaging, molecular profiling, cell count and apoptosis Markers.

“Origin” as referred to in ‘tissue of origin’ means either the tissue type (lung, colon, etc.) or the histological type (adenocarcinoma, squamous cell carcinoma, etc.) depending on the particular medical circumstances and will be understood by anyone of skill in the art. A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product. The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with an indication or tissue type. Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in for instance, 5445934; 5532128; 5556752; 5242974; 5384261; 5405783; 5412087; 5424186; 5429807; 5436327; 5472672; 5527681; 5529756; 5545531; 5554501; 5561071; 5571639; 5593839; 5599695; 5624711; 5658734; and 5700637.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in 6271002; 6218122; 6218114; and 6004755.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The selection can be based on statistical tests that produce ranked lists related to the evidence of significance for each gene's differential expression between factors related to the tumor's original site of origin. Examples of such tests include ANOVA and Kruskal-Wallis. The rankings can be used as weightings in a model designed to interpret the summation of such weights, up to a cutoff, as the preponderance of evidence in favor of one class over another. Previous evidence as described in the literature may also be used to adjust the weightings.

A preferred embodiment is to normalize each measurement by identifying a stable control set and scaling this set to zero variance across all samples. This control set is defined as any single endogenous transcript or set of endogenous transcripts affected by systematic error in the assay, and not known to change independently of this error. All Markers are adjusted by the sample specific factor that generates zero variance for any descriptive statistic of the control set, such as mean or median, or for a direct measurement. Alternatively, if the premise of variation of controls related only to systematic error is not true, yet the resulting classification error is less when normalization is performed, the control set will still be used as stated. Non-endogenous spike controls could also be helpful, but are not preferred.

Gene expression profiles can be displayed in a number of ways. The most common is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (down-regulation) appears in the blue portion of the spectrum while a ratio greater than one (up-regulation) appears in the red portion of the spectrum. Commercially available computer software programs are available to display such data including “Genespring” (Silicon Genetics, Inc.) and “Discovery” and “Infer” (Partek, Inc.)

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

Modulated genes used in the methods of the invention are described in the Examples. The genes that are differentially expressed are either up regulated or down regulated in patients with carcinoma of a particular origin relative to those with carcinomas from different origins. Up regulation and down regulation are relative terms meaning that a detectable difference (beyond the contribution of noise in the system used to measure it) is found in the amount of expression of the genes relative to some baseline. In this case, the baseline is determined based on the algorithm. The genes of interest in the diseased cells are then either up regulated or down regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of relapse, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic Markers, it is often desirable to use the fewest number of Markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Markowitz (1952). Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional Markers such as serum protein Markers (e.g., Cancer Antigen 27.29 (“CA 27.29”)). A range of such Markers exists including such analytes as CA 27.29. In one such method, blood is periodically taken from a treated patient and then subjected to an enzyme immunoassay for one of the serum Markers described above. When the concentration of the Marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

The present invention provides a method for analyzing a biological specimen for the presence of cells specific for an indication by: a) enriching cells from the specimen; b) isolating nucleic acid and/or protein from the cells; and c) analyzing the nucleic acid and/or protein to determine the presence, expression level or status of a Biomarker specific for the indication.

The biological specimen can be any known in the art including, without limitation, urine, blood, serum, plasma, lymph, sputum, semen, saliva, tears, pleural fluid, pulmonary fluid, bronchial lavage, synovial fluid, peritoneal fluid, ascites, amniotic fluid, bone marrow, bone marrow aspirate, cerebrospinal fluid, tissue lysate or homogenate or a cell pellet. See, e.g. 20030219842.

The indication can include any known in the art including, without limitation, cancer, risk assessment of inherited genetic pre-disposition, identification of tissue of origin of a cancer cell such as a CTC 60/887,625, identifying mutations in hereditary diseases, disease status (staging), prognosis, diagnosis, monitoring, response to treatment, choice of treatment (pharmacologic), infection (viral, bacterial, mycoplasmal, fungal), chemosensitivity 7112415, drug sensitivity, metastatic potential or identifying mutations in hereditary diseases.

Cells enrichment can be by any method known in the art including, without limitation, by antibody/magnetic separation, (Immunicon, Miltenyi, Dynal) 6602422, 5200048, fluorescence activated cell sorting, (FACs) 7018804, filtration or manually. The manual enrichment can be for instance by prostate massage. Goessl et al. (2001) Urol 58:335-338.

The nucleic acid can be any known in the art including, without limitation, is nuclear, mitochondrial (homeoplasmy, heteroplasmy), viral, bacterial, fungal or mycoplasmal.

Methods of isolating nucleic acid and protein are well known in the art. See e.g. 6992182, RNA www.ambion.com/techlib/basics/rnaisol/index.html, and 20070054287.

DNA analysis can be any known in the art including, without limitation, methylation—de-methylation, karyotyping, ploidy (aneuploidy, polyploidy), DNA integrity (assessed through gels or spectrophotometry), translocations, mutations, gene fusions, activation—de-activation, single nucleotide polymorphisms (SNPs), copy number or whole genome amplification to detect genetic makeup. RNA analysis includes any known in the art including, without limitation, q-RT-PCR, miRNA or post-transcription modifications. Protein analysis includes any known in the art including, without limitation, antibody detection, post-translation modifications or turnover. The proteins can be cell surface markers, preferably epithelial, endothelial, viral or cell type. The Biomarker can be related to viral/bacterial infection, insult or antigen expression.

The claimed invention can be used for instance to determine metastatic potential of a cell from a biological specimen by isolating nucleic acid and/or protein from the cells; and analyzing the nucleic acid and/or protein to determine the presence, expression level or status of a Biomarker specific for metastatic potential.

The cells of the claimed invention can be used for instance to identify mutations in hereditary diseases cell from a biological specimen by isolating nucleic acid and/or protein from the cells; and analyzing the nucleic acid and/or protein to determine the presence, expression level or status of a Biomarker specific for specific for a hereditary disease.

The cells of the claimed invention can be used for instance to obtain and preserve cellular material and constituent parts thereof such as nucleic acid and/or protein. The constituent parts can be used for instance to make tumor cell vaccines or in immune cell therapy. 20060093612, 20050249711.

Kits made according to the invention include formatted assays for determining the gene expression profiles. These can include all or some of the materials needed to conduct the assays such as reagents and instructions and a medium through which Biomarkers are assayed.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The present invention defines specific marker portfolios that have been characterized to detect a single circulating breast tumor cell in a background of peripheral blood. The molecular characterization multiplex assay portfolio has been optimized for use as a QRT-PCR multiplex assay where the molecular characterization multiplex contains 2 tissue of origin markers, 1 epithelial marker and a housekeeping marker. QRT-PCR will be carried out on the Smartcycler II for the molecular characterization multiplex assay. The molecular characterization singlex assay portfolio has been optimized for use as a QRT-PCR assay where each marker is run in a single reaction that utilizes 3 cancer status markers, 1 epithelial marker and a housekeeping marker. Unlike the RPA multiplex assay the molecular characterization singlex assay will be run on the Applied Biosystems (ABI) 7900HT and will use a 384 well plate as it platform. The molecular characterization multiplex assay and singlex assay portfolios accurately detect a single circulating epithelial cell enabling the clinician to predict recurrence. The molecular characterization multiplex assay utilizes Thermus thermophilus (TTH) DNA polymerase due to its ability to carry out both reverse transcriptase and polymerase chain reaction in a single reaction. In contrast, the molecular characterization singlex assay utilizes the Applied Biosystems One-Step Master Mix which is a two enzyme reaction incorporating MMLV for reverse transcription and Taq polymerase for PCR. Assay designs are specific to RNA by the incorporation of an exon-intron junction so that genomic DNA is not efficiently amplified and detected.

The present invention demonstrates the method to capture the CTCs and culture them in vitro. The experiment and the results are described below.

There are several novel aspects of this invention. First, the invention is the first demonstration of the combination of multiplex qRTPCR assays and the CellSearch technology for enrichment of circulating epithelial cells. We provide detailed description on novel methods developed to isolate the RNA after enrichment and use of the RNA in a qRTPCR assay. Secondly, the invention can be used as a surrogate for the cells themselves. That is, in clinical settings where very small numbers of circulating cells are found or in situations where very few intact circulating cells are found (since damaged cells are not recognized by the CellSearch enumeration algorithm), the use of the qRTPCR assay could provide a more sensitive enumeration of circulating tumor cells because the RNA would be isolated from both intact and damaged circulating tumor cells, increasing sensitivity of the detection, and the highly sensitive qRTPCR assays could further increase sensitivity. A final key aspect of the invention is that, by using a quantitative multiplex assay, one may be able to generate an algorithm based on two or more genes to generate prognostic information on patients from whom one has isolated circulating tumor cells. Importantly, the molecular information may provide additional or even new prognostic information when combined with the enumeration of circulating epithelial cells.

In one embodiment, the Molecular characterization singlex assay is based on Quantitative Reverse Transcriptase Polymerase Chain Reaction (QRT-PCR) where each marker is run in an individual reaction. The present invention describes the use of 3 tissue of origin markers, 1 epithelial marker for confirmation that circulating tumor cells are present from breast cancer and a control marker for verification of sample quality. Specific primer/probe combinations for each marker are designed to result in high specificity and sensitivity analysis for predicting recurrence in breast cancer patients. These primer/probe combinations for specific markers are optimized for the Applied Biosystems (ABI) 7900HT platform to detect a single circulating breast tumor cell in a background of peripheral blood. Results from this assay show that it could be used in parallel with the CellSearch™ CTC Kit enumeration kit and thus is beneficial to both the clinician and patient for predicting recurrence.

In a second embodiment, the Molecular characterization breast multiplex assay is also based on QRT-PCR, however in contrast to the singlex assay presented above, patient sample is analyzed in a single reaction with 3 diagnostic markers enabling a higher percentage of detection. The present invention describes the use of 2 tissue of origin markers, 1 epithelial marker for confirmation that circulating tumor cells are present from breast cancer and a control marker for verification of sample quality. Specific primer/probe combinations for each marker are designed to result in high sensitivity while very specific in a background of peripheral blood leukocytes PBLs. Feasibility of these applications are demonstrated by the ability of this assay to detect <5 SKBR3 cells spiked into 7.5 ml of peripheral blood. These primer/probe combinations for specific markers are optimized for the Smartcycler II platform. The results from this assay present a method for detection of breast circulating tumor cells that is unmatched when compared to current available methods due to the assay sensitivity and simultaneous use of 4 genes. It will be our intention to makes this assay available for commercial use in conjunction with the CellSearch™ CTC enumeration kit.

In a third embodiment, the molecular characterization assay is based on qRTPCR for the characterization of circulating prostate cells. In this example, a very sensitive multiplex assay incorporates 1 epithelial marker (CK19), one prostate tissue of origin marker (PSA, also known as kallikrein 3), and one control gene (PBGD). This assay can also be used for the highly sensitive detection of prostate cells.

The present invention provides a method to culture CTCs from blood. The process involves use of CellSearch™ technology and its associated CellTracks® AutoPrep system and CellSearch™ Profile Kit. These propagated cells could be used in pharmacogenomic studies and also to extract the nucleic acids in sufficient quantities for use in molecular profiling studies. Finally, this assay could also be used as a confirmatory tool in combination with the enumeration results of CTCs.

The present invention provides a method to detect methylation markers in DNA from <5 cell equivalents (3 cells in this study) following the sodium bisulfite conversion. The process involves a pre-amplification of target region followed by a multiplex QMSP. There are several novel aspects of this invention. First, the invention is the first demonstration of the combination of multiplex QMSP (involving nested PCR) assays and its extension to the CellSearch™ technology that enriches the circulating tumor cells (CTCs). Secondly, QMSP assay may provide useful information on several molecular markers thus making it more sensitive when combined with CellSearch™ technology. Thirdly, multiplex QMSP assay may be able to provide a new prognostic method for multiple tumor cell detection, for example, prostate and breast cancers. Finally, this assay could also be used as a confirmatory tool in combination with the enumeration result of CTCs.

The present invention defines Methylation specific marker portfolios that have been characterized to detect <20 pg of DNA after Sodium Bisulfite Conversion, equivalent to 3 circulating tumor cell, in a background of peripheral blood Leukocyte (PBL), equivalent to 10,000 to 100,000 PBL. Currently, the molecular characterization multiplex contains 1 DNA Methylation Specific marker and a housekeeping marker (additional 2 of DNA Methylation Specific markers will be added soon). Genomic DNA will be subjected to sodium bisulfite conversion and purification using ZymoResearch Kit. A pre-amplification of target regions using nested primer sets (outer primers) will be carried out on a thermocycler. In a subsequent QMSP reaction, a fluorescent signal will be generated by using inner primers with a Scorpion probe design on Cepheid's Smartcycler® or equivalent platform.

TABLE I
PCR Primer sequences

| | |
|---|---|
| | SEQ |
| | ID |
| Sequences | NO: |

Outer Primers

| | | |
|---|---|---|
| GSTP1_332_U18 | TCGGGGATTTTAGGGCGT | 1 |
| GSTP1_513_L21 | ACGAAAACTACGACGACGAAA | 2 |
| Actin_309_U24 | GATATAAGGTTAGGGATAGGATAG | 3 |
| Actin_501_L22 | AACCAATAAAACCTACTCCTCC | 4 |

Inner Scorpion
probe/primer

| | | |
|---|---|---|
| GSTP1_Fam_Sc_ | FAMCGCACGGCGAACTCCCGCCGACGTGC | 5 |
| 1112_L15 | G BHQ-HEG-TGTAGCGGTCGTCGGGGTTG | |
| GSTPi_1151_L22 | 5′ GCCCCAATACTAAATCACGACG 3′ | 6 |
| Actin_Q670_Sc_ | Q670-CCGCGCATCACCACCCCACACGCGCG | 7 |
| 382_L15 | G-BHQ2-HEG-GGAGTATATAGGTTGGGGAA | |
| | GTTTG | |
| Actin_425_L27 | 5′ AACACACAATAACAAACACAAATTCA | 8 |
| | C 3′ | |

### Experimental Setup:

The first PCR (pre-amplification) reaction reagent formulations and cycling conditions as follows:

TABLE II
Reagents for pre-amplification PCR

| | Final | |
|---|---|---|
| Reaction Buffer | Concentration | |
| (NH₄)₂SO₄ | 16.6 | mM |
| Tris (pH 8.8) | 67 | mM |
| MgCl₂ | 6.7 | mM |
| β-mercaptoethanol | 10 | mM |
| Taq enzyme/Ag mix | | |
| Taq Polymerase | 5 | U/μl |
| TP6-25 antibody | 0.65 | mg/ml |
| Outer primer Mix | | |
| GSTP1 | 0.25 | μM |
| Actin | 0.15 | μM |
| DNTPs mix | 1.25 | mM |

TABLE III
Cycling conditions for pre-amplification

| Temperature (° C.) | Time | | Cycles |
|---|---|---|---|
| 94 | 2 | min | 1 |
| 92 | 20 | sec | 20-25 |
| 55 | 30 | sec | |
| 70 | 30 | sec | |
| 70 | 5 | min | 1 |

6-10% of first PCR product, as is with no purification, from above will be transferred to a fresh tube for 2^{nd} PCR with the addition of the following reagents (Table IV) and subject to the cycling conditions in Table V.

The second PCR reaction reagent formulations and cycling conditions as follows:

TABLE IV
Reagents for the 2^{nd} PCR

| Reaction Buffer | Final concentration | |
|---|---|---|
| (NH₄)₂SO₄ | 16.6 | mM |
| Tris (pH 8.8) | 67 | mM |
| MgCl₂ | 6.7 | mM |
| β-mercaptoethanol | 10 | mM |
| Taq enzyme/Ab mix | | |
| Taq Polymerase | 5 | U/μl |
| TP6-25 antibody | 0.65 | mg/ml |
| Inner Scorpion probe/primer | | |
| GSTP1 | 0.5 | μM |
| Actin | 0.3 | μM |
| dNTPs mix | 1.25 | mM |

TABLE V
Cycling conditions for 2^{nd} PCR

| Temperature (° C.) | Time | | cycles |
|---|---|---|---|
| 95 | 60 | sec | 1 |
| 95 | 30 | sec | 40 |
| 55 | 30 | sec | |
| 72 | 5 | min | 1 |

The following DNA samples were used in this study:

CpGenome Universal methylated DNA (CpG M), Prostate Adenocarcinoma DNA (PC) or Prostate Normal DNA (PN) in a background of spiked DNA (100 ng or 500 ng) from peripheral blood lymphocytes (PBL). QMSP reactions were carried out after sodium bisulfite conversion. The following examples are meant to illustrate but not limit the invention.

### EXAMPLE #1

### Gene Expression Analysis of Serially Diluted Breast RNA Spiked Into A Background of Leukocyte RNA

The assays from the molecular characterization singlex assay portfolio include a junction-specific PCR probe that eliminates amplification of genomic DNA. The primer and dual-labeled hydrolysis probe sequences tested for this sample are shown below:

RPA Singlex Assays

| | | |
|---|---|---|
| | | SEQ |
| | | ID |
| Assays | Sequence | NO: |

| | | |
|---|---|---|
| B305D-RPAU22 | AATGGCCAAAGCACTGCTCTTA | 9 |
| B305D-RPAL21 | ACTTGCTGTTTTTGCTCATGT | 10 |
| B305D-RPAFAMP30 | FAM-ATCGAATCAAAAAACAAGCATGGCCT | 11 |
| | CACA-BHQ1-TT | |
| CK19-RPAU22 | CACCCTTCAGGGTCTTGAGATT | 12 |
| CK19-RPAL20 | TCCGTTTCTGCCAGTGTGTC | 13 |
| CK19-RPAFAMP24 | FAM-ACAGCTGAGCATGAAAGCTGCCTT- | 14 |
| | BHQ1-TT | |
| PBGD-RPAU22 | CCACACACAGCCTACTTTCCAA | 15 |
| PBGD-RPAL21 | TACCCACGCGAATCACTCTCA | 16 |
| PBGD-RPAP27FAM | FAM-AACGGCAATGCGGCTGCAACGGCGGA | 17 |
| | A-BHQ1-TT | |
| MG-RPAU21 | AGTTGCTGATGGTCCTCATGC | 18 |
| MG-RPAL24 | CACTTGTGGATTGATTGTCTTGGA | 19 |
| MG-RPAP23FAM | FAM-CCCTCTCCCAGCACTGCTACGCA- | 20 |
| | BHQ1-TT | |
| P1B289U21 | GAGTACGTGGGCCTGTCTGCA | 21 |
| P1B360L21 | TTGCACTCCTTGGGGGTGACA | 22 |
| P1B311FAMP25 | FAM-ACCAGTGTGCCGTGCCAGCCAAGGA- | 23 |
| | BHQ1-TT | |

Each singlex reaction was carried out on the Applied Biosystems 7900HT using the following cycling conditions and reagent formulations as follows:

Cycling Conditions

48° C. × 30 min
95° C. × 10 min
40 cycles of
95° C. for 15 sec
60° C. for 1 min

| Reagent | FC | X1 (10 μl) |
|---|---|---|
| RT-PCR Master Mix | 1x | 5.00 |
| Multiscribe Enzyme | .25 U/μl | 0.25 |
| Primer/Probe Mix | 0.6 μM/0.25 μM | 1.00 |
| Sample | | 3.75 |
| Total | | 10.00 |

Following RNA isolation of SKBR3 and MCF7 breast cancer cell lines total RNA was serially diluted to represent 1-400 cell equivalents (CE). The serially diluted RNA was then spiked into a background leukocyte total RNA equivalent to 50,000CE. Quantitative Real-Time PCR was applied and results of optimal assays supporting this invention are shown below.

| | | RNA | | | 20 ng | | | |
|---|---|---|---|---|---|---|---|---|
| | Cell | serial | Dilutions | Spiked | PBL | gDNA | Leuk | NT |
| Assay | line | 20 ng | 2 ng | in 0.2 ng | 0.02 ng | 200 ng | 20 ng | water |
| B305D-RPA | MCF7 | 23.64 | 27.64 | 31.36 | 35.84 | 40.00 | 38.40 | 40.00 |
| | SKBR3 | 24.65 | 28.78 | 33.37 | 36.65 | | | |
| CK-19-RPA | MCF7 | 16.95 | 20.97 | 25.33 | 29.66 | 40.00 | 34.19 | 40.00 |
| | SKBR3 | 17.54 | 21.18 | 25.47 | 29.64 | | | |
| P1B-RPA | MCF7 | 22.69 | 26.38 | 30.64 | 34.50 | 40.00 | 39.09 | 40.00 |
| | SKBR3 | 25.18 | 28.87 | 32.78 | 36.59 | | | |
| PBGD-RPA | MCF7 | 22.73 | 26.57 | 30.55 | 34.73 | 40.00 | 25.54 | 40.00 |
| | SKBR3 | 23.59 | 27.03 | 31.01 | 35.49 | | | |
| MG-RPA | MCF7 | 31.91 | 36.58 | 40.00 | 39.02 | 40.00 | 40.00 | 40.00 |
| | SKBR3 | 23.07 | 27.34 | 31.34 | 35.58 | | | |

### EXAMPLE #2

### Gene Expression Analysis of Alternative Markers or Assays

Additional designs tested include a junction-specific PCR probe that eliminates amplification of genomic DNA. The primer and dual-labeled hydrolysis probe sequences tested for this sample are shown below:

RPA Multiplex Assays

| | | SEQ |
|---|---|---|
| | | ID |
| Assays | Sequence | NO: |
| PIP82U20 | CTCCTGGTTCTCTGCCTGCA | 24 |
| PIP155L24 | GACGTACTGACTTGGGAATGTCAA | 25 |
| PIP116P28 | FAM-AAGCTCAGGACAACACTCGGAAGATCAT- | 26 |
| | BHQ1-TT | |
| P1B284U22 | CTGAGGAGTACGTGGGCCTGTC | 27 |
| P1B360L21 | TTGCACTCCTTGGGGGTGACA | 28 |
| P1B308FAMP25 | FAM-CAAACCAGTGTGCCGTGCCAGCCAA- | 29 |
| | BHQ1-TT | |
| PIP-INT-U | GCTTGGTGGTTAAAACTTACC | 30 |
| PIP-INT-L | TGAACAGTTCTGTTGGTGTA | 31 |
| PIP-304-P27- | FAM-CTGCCTGCCTATGTGACGACAATCCGG- | 32 |
| FAM | BHQ1-TT | |
| HPRT (BHQ)- | TGACACTGGCAAAACAATGCA | 33 |
| 496F | | |
| HPRT (BHQ)- | GGTCCTTTTCACCAGCAAGCT | 34 |
| 589R | | |
| HPRT (BHQ)- | FAM-CTTTGCTTTCCTTGGTCAGGCAGTATAATC | 35 |
| 519T | CA-BHQ1-TT | |
| B305D-CC4-U | AAAAACAAGCATGGCCTAC | 36 |
| B305D-CC4-L | CAGCAAGTTGAGAGCAGTCCT | 37 |
| B305D-923- | FAM-CATGAGCAAAAACAGCAAGTCGTGAAATT- | 38 |
| P29-FAM | BHQ1-TT | |
| PDEF1024U20 | CGCCCACCTGGACATCTGGA | 39 |
| PDEF1087L23 | CACTGGTCGAGGCACAGTAGTGA | 40 |
| PDEF1045P25 | FAM-GTCAGCGGCCTGGATGAAAGAGCGG- | 41 |
| FAM | BHQ1-TT | |

Samples were prepared and transcripts amplified in the same manor as described in Example #1. Results of these alternative assays supporting this invention are shown below. When compared to the performance of markers in Example #1 the following results demonstrate assays that have inferior performance mostly contributed to lack of marker specificity and/or sensitivity and poor primer or probe design.

| | | | | Spiked in | | | |
|---|---|---|---|---|---|---|---|
| | | RNA Serial | Dilutions | 20 ng | PBL | Leuk | NT |
| Assay | Cell Line | 20 ng | 2 ng | 0.2 ng | 0.02 ng | 20 ng | water |
| PDEF-1024 | MCF7 | 24.90 | 28.99 | 33.09 | 36.49 | 33.10 | 40.00 |
| | SKBR3 | 22.85 | 26.72 | 30.89 | 35.57 | | |
| B305D-CC | MCF7 | 28.78 | 31.89 | 35.68 | 39.60 | 40.00 | 40.00 |
| | SKBR3 | 31.04 | 34.85 | 38.91 | 40.00 | | |
| P1B284 | MCF7 | 22.04 | 25.68 | 29.87 | 34.63 | 34.58 | 40.00 |
| | SKBR3 | 24.58 | 28.29 | 32.42 | 36.04 | | |
| HPRT496 | MCF7 | 23.93 | 27.38 | 31.58 | 35.12 | 26.98 | 40.00 |
| | SKBR3 | 25.13 | 29.07 | 33.30 | 36.95 | | |
| PIP82 | MCF7 | 35.77 | 40.00 | 40.00 | 40.00 | 38.75 | 40.00 |
| | SKBR3 | 27.88 | 31.48 | 35.67 | 38.49 | | |
| PIPINT | MCF7 | 35.22 | 40.00 | 38.51 | 40.00 | 36.72 | 40.00 |
| | SKBR3 | 26.66 | 30.63 | 34.73 | 39.34 | | |

### EXAMPLE #3

### QRT-PCR Analysis of Enriched SKBR3 and MCF7 Cells

The molecular characterization assay will combine the cell capture portion of CellSearch technology with a molecular detection assay. The sensitivity of the CellSearch assay may be improved by utilizing a molecular detection technology capable of detecting marker expression in both intact cells and cell fragments typically not called positive by the CellSearch assay. Isolation of RNA using immunomagnetically enriched SKBR3 and MCF7 cells spiked into healthy donor blood drawn into EDTA anticoagulant blood tubes was carried out as shown below.

| | | 25 CTC | 12.5 CTC | 1.25 CTC | 0 CTC | PC 1000 CTC | |
|---|---|---|---|---|---|---|---|
| Assay | Cell Line | (0.5 ng) | (0.25 ng) | (0.025 ng | (Leuk Bkgd) | (20 ng) | NC |
| B305D-RPA | SKBR3 | 33.75 | 36.40 | 37.59 | 40.00 | 26.34 | 40.00 |
| | MCF7 | 35.17 | 36.72 | 37.66 | 40.00 | 26.11 | 40.00 |
| CK19-RPA | SKBR3 | 24.76 | 27.56 | 30.00 | 40.00 | 19.20 | 40.00 |
| | MCF7 | 27.00 | 28.39 | 30.98 | 32.49 | 18.33 | 40.00 |
| MG-RPA | SKBR3 | 29.84 | 35.51 | 36.06 | 40.00 | 24.58 | 40.00 |
| | MCF7 | 39.19 | 38.42 | 35.87 | 35.70 | 24.42 | 40.00 |
| P1B-RPA | SKBR3 | 30.73 | 33.30 | 34.22 | 40.00 | 26.54 | 40.00 |
| | MCF7 | 35.84 | 35.61 | 40.00 | 37.40 | 26.75 | 40.00 |
| PBGD-RPA | SKBR3 | 27.42 | 27.10 | 28.99 | 32.01 | 25.81 | 40.00 |
| | MCF7 | 30.79 | 33.04 | 33.00 | 30.59 | 25.76 | 40.00 |

Feasibility of molecular characterization singlex assay has been demonstrated by sensitivity and reproducible detection of specific mRNA transcripts in <5 SKBR3 cells when enriched from 7.5 ml of healthy donor blood.

### EXAMPLE 4

### Molecular Characterization Multiplex Assay Analysis of Serially Diluted Breast RNA Spiked Into A Background of Leukocyte RNA

The assays from the RPA multiplex assay portfolio include a junction-specific PCR probe that eliminates amplification of genomic DNA. The primer and dual-labeled hydrolysis probe sequences tested for this sample are shown below:

RPA Multiplex

| | | SEQ |
|---|---|---|
| | | ID |
| Assay | Sequence | NO: |
| B305D-RPAU22 | AATGGCCAAAGCACTGCTCTTA | 42 |
| B305D-RPAL21 | ACTTGCTGTTTTTGCTCATGT | 43 |
| B305D- | TR-ATCGAATCAAAAAACAAGCATGGCCTCACA- | 44 |
| RPATRP30 | BHQ2-TT | |
| CK19-RPAU22 | CACCCTTCAGGGTCTTGAGATT | 45 |
| CK19-RPAL20 | TCCGTTTCTGCCAGTGTGTC | 46 |
| CK19- | CY3-ACAGCTGAGCATGAAAGCTGCCTT-BHQ2- | 47 |
| RPACY3P24 | TT | |
| PBGD-RPAU22 | CCACACACAGCCTACTTTCCAA | 48 |
| PBGD-RPAL21 | TACCCACGCGAATCACTCTCA | 49 |
| PBGD- | CY5-AACGGCAATGCGGCTGCAACGGCGGAA- | 50 |
| RPACY5P27 | BHQ2-TT | |
| MG-RPAU21 | AGTTGCTGATGGTCCTCATGC | 51 |
| MG-RPAL24 | CACTTGTGGATTGATTGTCTTGGA | 52 |
| MG- | FAM-CCCTCTCCCAGCACTGCTACGCA-BHQ1- | 53 |
| RPAP23FAM | TT | |

Each multiplex reaction was carried out on the Smartcycler II using the following cycling conditions and reagent formulations as follows:

Cycling Conditions

95 C. × 3 sec
59 C. × 12 min
70 C. × 90 sec
40 cycles of:
95 C. for 20 sec
62 C. for 30 sec

| Reagents | FC | | X1 (25 ul) |
|---|---|---|---|
| 2.5x BLN Enzyme Mix | 1x | | 10 |
| Tth Polymerase | 6.5 | U | |
| 0.13 mg/ml TP6-25AB | 0.052 | mg/ml | |
| 2.5 x Base BLN master Mix | 1x | | 9 |
| 7.5 mM MnSo4 | 3 | mM | |
| 3.125 mM MgCl | 1.25 | mM | |
| 0.5 mM dNTP | 0.2 | mM | |
| 25X Primer Mix | 1x | | 1 |
| 11.25 uM F & R/5 uM P MG | 0.45/0.2 | uM | |
| 11.25 uM F & R/5 uM P Ck19 | 0.45/0.2 | uM | |
| 11.25 uM F & R/5 uM P B305D | 0.45/0.2 | uM | |
| 7.5 uM F & R/5 uM P PBGD | 0.3/0.2 | uM | |
| 375 mM (NH4)2SO4 | 15 | mM | 1 |
| Sample | | | 5 |
| Total | | | 25 |

Following RNA isolation of SKBR3 breast cancer cell lines, total RNA was serially diluted to represent 1-125 cell equivalents (CE). The serially diluted RNA was then spiked into a background leukocyte total RNA equivalent to 50,000CE. Quantitative Real-Time PCR was applied and results supporting this invention are shown below.

| | | RNA Serial Dilutions Spiked | | | | | |
|---|---|---|---|---|---|---|---|
| | Cell | in 20 ng PBL | | | | Leuk | NT |
| Assay | Line | 2.5 ng | 0.5 ng | 0.1 ng | 0.02 ng | 20 ng | water |
| MG- | SKBR3 | 26.45 | 28.55 | 31.00 | 32.80 | 0.00 | 40.00 |
| RPA | | | | | | | |
| CK19- | SKBR3 | 17.54 | 23.95 | 26.25 | 28.55 | 37.35 | 40.00 |
| RPA | | | | | | | |
| B305D- | SKBR3 | 24.65 | 29.25 | 30.70 | 34.25 | 39.55 | 40.00 |
| RPA | | | | | | | |
| PBGD- | SKBR3 | 27.90 | 28.40 | 29.15 | 28.85 | 29.10 | 40.00 |
| RPA | | | | | | | |

### EXAMPLE #5

### RPA Multiplex QRT-PCR Analysis of Enriched SKBR3 cells

Molecular characterization Multiplex assay will combine the cell capture portion of CellSearch technology with a molecular detection assay. The sensitivity of the CellSearch assay may be improved by utilizing a molecular detection technology capable of detecting marker expression in both intact cells and cell fragments typically not called positive by the CellSearch assay. Isolation of RNA using immunomagnetically enriched SKBR3 cells transcribing only CK19 spiked into healthy donor blood drawn into EDTA anticoagulant blood tubes was carried out as shown below. In contrast to the molecular characterization Singlex assay where a patient sample has to be divided between all reactions, the molecular characterization Multiplex assay offers increased sensitivity by enabling the user to analyze the molecular profile of an entire sample in a single reaction.

| | | 500 CTC | 50 CTC | 5 CTC | 0 CTC | |
|---|---|---|---|---|---|---|
| Assay | Cell Line | (10 ng) | (1 ng) | (0.1 ng) | (Leuk | NC |
| CK19-RPA | SKBR3 | 26.60 | 29.10 | 36.30 | 40.00 | 40.00 |
| PBGD-RPA | SKBR3 | 28.80 | 30.80 | 33.20 | 36.05 | 40.00 |

### EXAMPLE #6

### RNA Stability Analysis of Enriched SKBR3 Cells

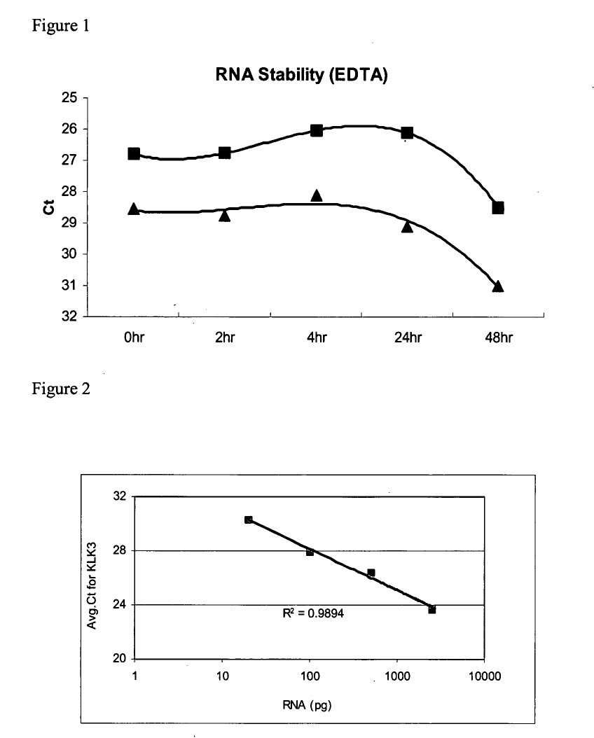

RNA stability of intracellular RNA was evaluated through QRT-PCR using the molecular characterization Multiplex assay over a 48-hour time course. 200 SKBR3 cells were spiked into multiple tubes of 7.5 ml of healthy donor blood. At the end of each time point samples were processed using the cell capture portion of CellSearch technology and the CellSearch Profile Kit. After RNA isolation samples were analyzed and results are shown in the table below and FIG. 1.

| | | 0 hr | | | | | |
|---|---|---|---|---|---|---|---|
| Assay | Cell Line | NTC | 0 hr | 2 hr | 4 hr | 24 hr | 48 hr |
| CK19-RPA | SKBR3 | 0.00 | 26.80 | 26.75 | 26.05 | 26.10 | 28.50 |
| PBGD-RPA | SKBR3 | 30.90 | 28.55 | 28.75 | 28.10 | 29.10 | 31.00 |

The present invention provide methods, apparatus and kits for sample processing of circulating tumor cells (CTC) within peripheral blood and assessing their gene expression profiles while providing support for the Cell search platform for disease recurrence testing. Examples show the ability to detect a single circulating tumor cells in a background of peripheral blood using a novel multiplex assay that offers increased advantages over traditional singlex RT-PCR assays.

### EXAMPLE #7

### Prostate Circulating Cells

Prostate RNA was spiked into RNA from leukocytes and tested in a multiplex assay on the Cepheid Smartcycler II. Representative data is shown below and in FIG. 2.

| | Average Ct value | | |
|---|---|---|---|
| prostate RNA (pg) | PBGD w/PBL | KLK3 w/PBL | CK19 w/PBQL |
| 2500 | 29.2 | 23.7 | 27.9 |
| 500 | 29.1 | 26.4 | 29.9 |
| 100 | 29.3 | 27.9 | 31.8 |
| 20 | 29.9 | 30.3 | 34.5 |
| 0 (20 ng PBL RNA | 28.8 | 40.0 | 36.7 |
| only) | | | |

### EXAMPLE #7

### Increased Sensitivity for Gene Expression Analysis using Breast RPA Nested QRT-PCR Multiplex Assay

Single-round real-time reverse transcription (RT)-PCR detection is generally inconsistent because the concentration of extracted RNA from circulating tumor cells is often very low. Two-round QRT-PCR using nested primers enhances both the specificity and sensitivity of the assay specifically those working with low or poor quality target or rare messages. This method incorporates two pairs of primers that are used to amplify first a larger template nucleic acid

RPA Nested Primers

| | |
|---|---|
| Assay | Sequence |

| | |
|---|---|
| B305D1223U25 | TAATGTTGCTGGAACATGGCACTGA |
| B305D1448L26 | TCTTCCATATCTATCCAGCGCATTTA |
| CK19 901U21 | AGATGAGCAGGTCCGAGGTTA |
| CK19 1094L23 | CCTGATTCTGCCGCTCACTATCA |
| PBGD107U21 | GGACCTTAGCGGCACCCACAC |
| PBGD240L22 | CTGTCCGTCTGTATGCGAGCAA |
| MG39U20 | CACCGACAGCAGCAGCCTCA |
| MG148L24 | CACTTGTGGATTGATTGTCTTGGA |

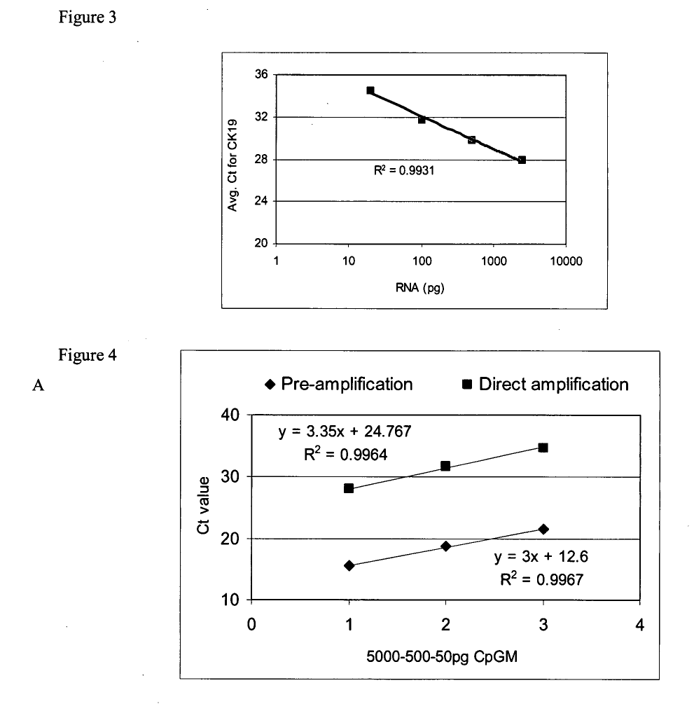

molecule and, subsequently, a target nucleic acid sequence that is contained in the amplified template molecule. Thus by employing two-round QRT-PCR both sensitivity and specificity are increased for the breast RPA molecular companion assay. FIG. 3.

Nested multiplex amplification reaction was carried out on the Smartcycler II using the following cycling conditions and reagent formulations as follows: As described above, serially diluted SKBR3 RNA spiked into a background of leukocyte total RNA was used in the following example.

| Reagents | FC | | X1 (25 ul) |
|---|---|---|---|
| BLN Enzyme Mix | 1X | | 2.5 |
| Tth Polymerase | 6.5 | U | |
| TP6-25 AB | 0.052 | mg/ml | |
| 2.5x BLN Master Mix | 1X | | 10 |
| 7.5 mM MnSO4 | 3 | mM | |
| 3.125 mM MgCl | 1.25 | mM | |
| 0.5 mM dNTP | 0.2 | mM | |
| 25X Primer Mix | 1X | | 1 |
| 5 uM F&R MG | 0.2 | uM | |
| 11.25 uM F&R Ck19 | 0.45 | uM | |
| 11.25 uM F&R B305D | 0.45 | uM | |
| 7.5 uM F&R PBGD | 0.5 | uM | |
| 375 mM (NH4)2SO4 | 15 | mM | 1 |
| Sample | | | 10.5 |
| Total | | | 25 |

| Temperature | Time | |
|---|---|---|
| 95 C. | 3 | sec |
| 59 C. | 12 | min |
| 70 C. | 90 | sec |

15 Cycles

| | | |
|---|---|---|
| 95 C. | 20 | sec |
| 62 C. | 30 | sec |

Following first round amplification, tubes are spun and a three micro liter aliquot is drawn from the first tube and expelled into a second tube containing the following primers, probes and reagents.

RPA Multiplex Assays

| | |
|---|---|
| Assays | Sequence |

| | |
|---|---|
| B305D-RPAU22 | AATGGCCAAAGCACTGCTCTTA |
| B305D-RPAL21 | ACTTGCTGTTTTTGCTCATGT |
| B305D-RPATRP30 | TR-ATCGAATCAAAAAACAAGCATGGCCTCACA- |
| | BHQ2-TT |
| CK19-RPAU22 | CACCCTTCAGGGTCTTGAGATT |
| CK19-RPAL20 | TCCGTTTCTGCCAGTGTGTC |
| CK19-RPACY3P24 | CY3-ACAGCTGAGCATGAAAGCTGCCTT-BHQ2-TT |
| PBGD-RPAU22 | CCACACACAGCCTACTTTCCAA |
| PBGD-RPAL21 | TACCCACGCGAATCACTCTCA |
| PBGD-RPACY5P27 | CY5-AACGGCAATGCGGCTGCAACGGCGGAA- |
| | BHQ2-TT |
| MG-RPAU21 | AGTTGCTGATGGTCCTCATGC |
| MG-RPAL24 | CACTTGTGGATTGATTGTCTTGGA |
| MG-RPAP23FAM | FAM-CCCTCTCCCAGCACTGCTACGCA-BHQ1-TT |

Quantitative Real-Time PCR was applied using the following parameters and results supporting this invention are shown below.

| Temperature | Time | |
|---|---|---|
| 95 C. | 3 | sec |
| 59 C. | 12 | min |
| 70 C. | 90 | sec |

40 Cycles

| | | |
|---|---|---|
| 95 C. | 20 | sec |
| 62 C. | 30 | sec |

### EXAMPLE #8

| Reagents | FC | | X1 (25 ul) |
|---|---|---|---|
| BLN Enzyme Mix | 1X | | 2.5 |
| Tth Polymerase | 6.5 | U | |
| TP6-25 AB | 0.052 | mg/ml | |
| 2.5x BLN Master Mix | 1X | | 10 |
| 7.5 mM MnSO4 | 3 | mM | |
| 3.125 mM MgCl | 1.25 | mM | |
| 0.5 mM dNTP | 0.2 | mM | |
| 25X Primer Mix | 1X | | 1 |
| 5 uM F & R/2.5 uM P MG | 0.2/0.1 | uM | |
| 11.25 uM F & R/5 uM P Ck19 | 0.45/0.2 | uM | |
| 11.25 uM F & R/5 uM P B305D | 0.45/0.2 | uM | |
| 7.5 uM F & R/5 uM P PBGD | 0.3/0.2 | uM | |
| 375 mM (NH4)2SO4 | 15 | mM | 1 |
| Sample | | | 10.5 |
| Total | | | 25 |

Two Round RT-PCR

| Sample | MG | CK19 | B305D | PBGD |
|---|---|---|---|---|
| 2000 pg | 14.40 | 19.05 | 20.80 | 19.15 |
| 200 pg | 18.30 | 23.85 | 24.45 | 21.85 |
| 20 pg | 19.95 | 26.25 | 26.10 | 22.00 |
| Leuk | 34.90 | 35.75 | 28.55 | 21.85 |
| NT | 40.00 | 38.10 | 38.20 | 40.00 |

### Two Round Breast RPA Nested QRT-PCR Analysis of Enriched SKBR3 Cells

Breast RPA nested QRT-PCR multiplex assay will be used in conjunction with the CellSearch enrichment to improve molecular detection technology capable of detecting marker expression in both intact cells and cell fragments typically not called positive by the CellSearch CTC assay. Isolation of RNA using immunomagnetically enriched SKBR3 cells spiked into healthy donor blood drawn into EDTA anticoagulant blood tubes was carried out as shown below. In contrast to the one round RPA QRT-PCR assay where sensitivity and specificity are low, the two round breast RPA nested QRT-PCR offers increased sensitivity and specificity by enabling the user to have near single copy sensitivity.

Breast RPA Spike In

| Sample ID | SKBR3 | MG | B305D | PBGD |
|---|---|---|---|---|
| 1 | No Cells | 0.00 | 39.10 | 23.20 |
| 2 | No Cells | 37.60 | 36.60 | 24.20 |
| 3 | 5 cells | 26.00 | 27.20 | 22.80 |
| 4 | 5 cells | 25.40 | 29.30 | 25.80 |
| 5 | 50 cells | 37.40 | 29.70 | 22.50 |
| 6 | 50 cells | 23.70 | 28.20 | 22.20 |
| 7 | 500 cells | 18.60 | 24.30 | 20.30 |
| 8 | 500 cells | 21.60 | 25.50 | 22.30 |
| 10 | PC: 2 ng | 20.00 | 26.30 | 24.10 |
| 11 | NT | 35.30 | 0.00 | 37.50 |

### EXAMPLE #9

### Increased Sensitivity for Gene Expression Analysis using Prostate MCA Nested QRT-PCR Multiplex Assay

The need for improved sensitivity and specificity in PCR reactions designed to amplify rare sequences in circulating prostate tumor cells is addressed in the present invention. Technology utilized in the breast RPA nested QRT-PCR multiplex assay was crossed over to create the prostate nested QRT-PCR molecular companion assay (MCA).

Prostate MCA Nested Primers

| | |
|---|---|
| Assay | Sequence |

| | |
|---|---|
| KLK3;189U20 | TGCGGCGGTGTTCTGGTGCA |
| KLK3;294L24 | GACCTGAAATACCTGGCCTGTGTC |
| CK19 901U21 | AGATGAGCAGGTCCGAGGTTA |
| CK19 1094L23 | CCTGATTCTGCCGCTCACTATCA |
| PBGD107U21 | GGACCTTAGCGGCACCCACAC |
| PBGD240L22 | CTGTCCGTCTGTATGCGAGCAA |

Nested multiplex amplification reaction was carried out on the Smartcycler II using the following cycling conditions and reagent formulations as follows: As described above, serially diluted LNCAP RNA spiked into a background of leukocyte total RNA was used in the following example.

| Reagents | FC | | X1 (25 ul) |
|---|---|---|---|
| BLN Enzyme Mix | 1X | | 2.5 |
| Tth Polymerase | 6.5 | U | |
| TP6-25 AB | 0.052 | mg/ml | |
| 2.5x BLN Master Mix | 1X | | 10 |
| 7.5 mM MnSO4 | 3 | mM | |
| 3.125 mM MgCl | 1.25 | mM | |
| 0.5 mM dNTP | 0.2 | mM | |
| 25X Primer Mix | 1X | | 1 |
| 2.5 uM F & R KLK3 | 0.1 | uM | |
| 11.25 uM F & R Ck19 | 0.45 | uM | |
| 7.5 uM F & R PBGD | 0.3 | uM | |
| 375 mM (NH4)2SO4 | 15 | mM | 1 |
| Sample | | | 10.5 |
| Total | | | 25 |

| Temperature | Time | |
|---|---|---|
| 95 C. | 3 | sec |
| 59 C. | 12 | min |
| 70 C. | 90 | sec |

15 Cycles

| | | |
|---|---|---|
| 95 C. | 20 | sec |
| 62 C. | 30 | sec |

Following first round amplification, tubes are spun and a three micro liter aliquot is drawn from the first tube and expelled into a second tube containing the following primers, probes and reagents.

Prostate MCA Multiplex Assays

| | |
|---|---|
| Assays | Sequence |

| | |
|---|---|
| KLK3;209U19 | CCCCCAGTGGGTCCTCACA |
| KLK3;269L22 | AGGATGAAACAAGCTGTGCCGA |
| KLK3;242P26FAM | FAM-CAGGAACAAAAGCGTGATCTTGCTGG-BHQ1- |
| | TT |
| CK19-RPAU22 | CACCCTTCAGGGTCTTGAGATT |
| CK19-RPAL20 | TCCGTTTCTGCCAGTGTGTC |
| CK19-RPAFAMP24 | FAM-ACAGCTGAGCATGAAAGCTGCCTT-BHQ1-TT |
| PBGD-RPAU22 | CCACACACAGCCTACTTTCCAA |
| PBGD-RPAL21 | TACCCACGCGAATCACTCTCA |
| PBGD-RPAP27FAM | FAM-AACGGCAATGCGGCTGCAACGGCGGAA- |
| | BHQ1-TT |

Quantitative Real-Time PCR was applied using the following parameters and results supporting this invention are shown below.

| Temperature | Time | |
|---|---|---|
| 95 C. | 3 | sec |
| 59 C. | 12 | min |
| 70 C. | 90 | sec |

40 Cycles

| | | |
|---|---|---|
| 95 C. | 20 | sec |
| 58 C. | 30 | sec |

| Reagents | FC | | X1 (25 ul) |
|---|---|---|---|
| BLN Enzyme Mix | 1X | | 2.5 |
| Tth Polymerase | 6.5 | U | |
| TP6-25 AB | 0.052 | mg/ml | |
| 2.5x BLN Master Mix | 1X | | 10 |
| 7.5 mM MnSO4 | 3 | mM | |
| 3.125 mM MgCl | 1.25 | mM | |
| 0.5 mM dNTP | 0.2 | mM | |
| 25X Primer Mix | 1X | | 1 |
| 2.5 uM F & R/2.5 uM P KLK3 | 0.1/0.1 | uM | |
| 11.25 uM F & R/5 uM P Ck19 | 0.45/0.2 | uM | |
| 7.5 uM F & R/5 uM P PBGD | 0.3/0.2 | uM | |
| 375 mM (NH4)2SO4 | 15 | mM | 1 |
| Sample | | | 10.5 |
| Total | | | 25 |

Two Round RT-PCR

| Sample | | KLK3 | CK19 | PBGD |
|---|---|---|---|---|
| 20000 | pg | 9.80 | 16.20 | 20.10 |
| 200 | pg | 17.50 | 23.35 | 21.60 |
| 20 | pg | 20.10 | 25.30 | 21.80 |

| | | | |
|---|---|---|---|
| Leuk | 28.50 | 0.00 | 20.20 |
| NT | 0.00 | 38.90 | 0.00 |

### EXAMPLE #10

### Prostate MCA Multiplex QRT-PCR Analysis of Enriched LNCAP Cells

Prostate MCA nested QRT-PCR multiplex assay will be used in conjunction with the CellSearch enrichment to improve molecular detection technology capable of detecting marker expression in both intact cells and cell fragments typically not called positive by the CellSearch CTC assay. Isolation of RNA using immunomagnetically enriched LNCAP cells spiked into healthy donor blood drawn into EDTA anticoagulant blood tubes was carried out as shown below. In contrast to the one round prostate QRT-PCR assay where sensitivity and specificity are low, the two round prostate MCA nested QRT-PCR offers increased sensitivity and specificity by enabling the user to have near single copy sensitivity.

### EXAMPLE 11

### Spike-in of SKBR3 Followed by Capture by CellSearch™ System

SKRB3 cells were spiked at 1000 cells into 7.5 mL of donor blood (purple top Vacutainer® with EDTA as preservative) as shown in Table I.

TABLE I
Spiking of SKBR3 cell lines into donor blood

| Sample # | Donor | Conditions | Bar code # |
|---|---|---|---|
| 1 | 1 | Spiked w/ 1000 SKBr3 cells | V22166 |
| 2 | 1 | Spiked w/ 1000 SKBr3 cells | V22167 |
| 3 | 1 | Unspiked | V22168 |
| 4 | 1 | Unspiked | V22169 |
| 5 | 2 | Spiked w/ 1000 SKBr3 cells | V22170 |
| 6 | 2 | Spiked w/ 1000 SKBr3 cells | V22171 |

The CTCs were captured by EpCAM conjugated immunomagnetic beads using CellSearch™ Profile Kit and CellTracks® AutoPrep system. The tubes containing the captured CTCs were removed from the system and placed in Magcellect® magnet, incubated for 10 min. The supernatant was removed with the tube still in Magcellect®. The pellet was suspended in 200 μL of phosphate buffered saline (PBS). The cell suspension was plated into a 48-well plate containing 1.0 mL per well of complete Eagle's Minimal Essential Medium with 10% fetal bovine serum (FBS). The cells were qualitatively assessed for up to 14 days during the growth and the results of the observation are summarized in Table II. At the end of the culture period (5 days and 14 days), the cells were washed twice with PBS and lysed directly in the well using RLT buffer (Qiagen). Total RNA from the lysates was isolated by using RNeasy Micro Kit (Qiagen). These RNA samples will be used for further analyses including global gene expression.

### Results:

The CTCs captured using CellTracks® AutoPrep system seem to be viable although a decrease in doubling rate compared to the parental cells was observed

The leukocytes die off within 2 days of culture thus not interfering with the CTC growth

The CTCs were seen to be dividing although slower than the control parental cells as expected

The doubling time of growth for the CTCs was about >2× compared to parental cells

A difference in growth properties (quality and viability) was observed between the 2 replicates suggesting a possible effect from the donor blood

TABLE II
Qualitative Results

| Donor | Day 1 | Day 3 | Day 4 | Day 5 | Day 6 | Day 7 | Day 10 | Day 12 | Day 14 |
|---|---|---|---|---|---|---|---|---|---|
| #1 | ++++ | ++++ | ++++ | +++ | ++ | ++ | + | | |
| #2 | ++++ | ++++ | ++++ | ++++ | ++++ | +++ | +++ | ++ | ++ |
| Control | ++++++ | ++++++ | ++++++ | ++++++ | ++++++ | ++++++ | ++++++ | ++++++ | ++++++ |

Wells plated from cells that went through the CellTracks ® AutoPrep were never as dense as the control well.

### EXAMPLE 12

### Variable Quantities of CpG M DNA spiked into 100 or 500 ng PBL DNA

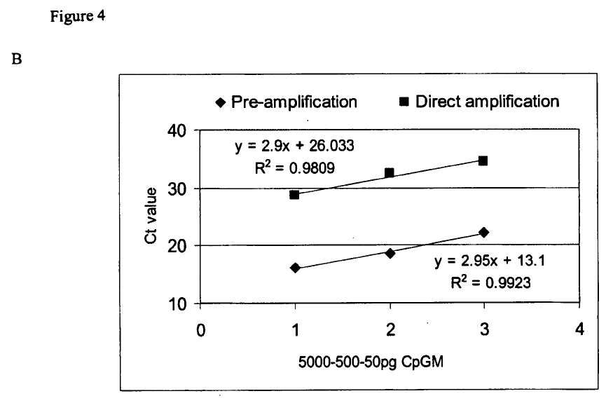

(25 cycles of pre-amplification PCR and use of 10% of diluted PCR product transferred to second PCR). Ct values for direct or pre-amplified template DNA (CpG M) are shown in the following table and FIG. 4.

| Pre-amplification (nested PCR) | | | No pre-amplification (directly PCR) | | |
|---|---|---|---|---|---|
| | Ct value | | | Ct value | |
| CpG M (pg) | Actin | GSTP1 | CpG M (pg) | Actin | GSTP1 |
| PBL-100 ng | | | PBL-100 ng | | |
| 5000 | 16.6 | 15.5 | 5000 | 27.6 | 28.0 |
| 500 | 16.2 | 18.8 | 500 | 27.8 | 31.7 |
| 50 | 15.8 | 21.5 | 50 | 27.7 | 34.7 |
| 0 | 5.3 | 0.0 | 0 | 27.5 | 0.0 |
| PBL-500 ng | | | PBL-500 ng | | |
| 5000 | 14.4 | 16.2 | 5000 | 25.3 | 28.7 |
| 500 | 13.8 | 18.7 | 500 | 26.1 | 32.3 |
| 50 | 14.0 | 22.1 | 50 | 25.3 | 34.5 |
| 0 | 3.2 | 0.0 | 0 | 25.4 | 0.0 |

### EXAMPLE 13

### PC and PN DNA spiked in 500 ng of PBL (Equivalent to 70,000 Cells)

(20 cycles of pre-amplification PCR followed by use of 6% of resultant product in the second PCR)

TABLE VII
Ct values for direct or pre-amplified template
DNA (prostate adenocarcinoma or normal)

| | Equivalent to Prostate | | |
|---|---|---|---|
| PC (pg) | cells | Actin (Ct) | GSTP1 (Ct) |
| 189 | 27 | 12.5 | 22.6 |
| 63 | 9 | 12.5 | 24.7 |
| 21 | 3 | 12.3 | 36.0 |
| 7 | 1 | 12.5 | 0.0 |
| PN | | | |
| 63 | 9 | 12.0 | 0.0 |
| PBL only | 0 | 12.1 | 0.0 |
| Neg (no DNA) | 0 | 0.0 | 0.0 |

### Results:

50 pg of CpG M DNA (equivalent to 7 cells) in a background of 100 ng or 500 ng of PBL (equivalent to 10,000 or 70,000 cells, respectively) was detected using QMSP with or without pre-amplification. Good linear response curves were generated for both pre-amplification and direct amplification reactions. In the initial study, <20 pg of DNA from prostate adenocarcinoma, equivalent to 3 circulating tumor cells, generated a signal specific to methylated GSTP1 region and was detected in a background of 70,000 PBL cells. On the other hand, no detectable signal from normal prostate (PN) or blood (PBL) DNA was observed suggesting the absence of methylated GSTP1. Nested QMSP sensitivity of detection of 1 copy in a background of 2.5×10⁴ copies (20 pg of methylated DNA in 500 ng of unmethylated DNA) is observed. No significant non-specific products were detected with nested QMSP method and the correct size of final PCR fragments were observed on the gel (data not shown). Further assay optimization experiments are underway to increase the detection sensitivity and to reduce the Ct value for <3 cells.

### EXAMPLE 14

### Demonstration of the Utility of the he Assay to Circulating Tumor Cells (CTCs) in Blood by Spiking Prostate Cancer Cell Lines (LnCAP and Du-145) into Donor Blood

Prostate tumor cell lines (LnCAP and DU-145) grown in culture were spiked at 30, 100, 300 and 500 cells into 7.5 ml of donor blood followed by capturing CTCs by CellTracks™ AutoPrep system of CellSearch™ platform using Profile Kit. Deoxyribonucleic acid from these cells was isolated using Qiagen microcolumns and subjected to bisulfite conversion reaction. The modified DNA from the last step was used in a 2 round q-MSP reaction using the conditions in Table III (22 cycles) and Table V. The results from the experiments are shown in Tables VIII and IX.

TABLE VIII
Ct values from CTCs (spiked cells, LnCAP)

| GSTP1 | 10% R1 transferred to R2 | | 0.2% R1 transferred to R2 | |
|---|---|---|---|---|
| LnCAP cell # | Ct | rfu | Ct | rfu |
| 0 (no spike) | 0.0 | −51 | 0.0 | −14 |
| 30 | 20.2 | 789 | 24.2 | 724 |
| 100 | 19.1 | 751 | 23.2 | 693 |
| 300 | 17.3 | 734 | 21.3 | 688 |
| Actin | 26.6-28.8 | 130-160 | 31.4-33.4 | 130-170 |

Differences of duplicated reactions were
less than 0.7Ct, except 30 cell which was 1.5-1.7 Ct.

| | | |
|---|---|---|
| Linear equation | Y = −1.0x + 22.83 | Y = −1.95x + 26.73 |
| | R² = 0.98 | R² = 0.99 |

TABLE IX
Ct values from CTCs (spiked cells, Du-145)
GSTP1

| Du-145 cell # | 10% R1 | | 50% R1 | |
|---|---|---|---|---|
| 0 (no spike) | 0 | 0 | 0 | 0 |
| 20 | 0 | 0 | 0 | 0 |
| 100 | 0 | 0 | 0 | 0 |
| 300 | 0 | 0 | 0 | 0 |
| 500 | 27.6 | 199 | 20.6 | 142 |
| 500 cell ctrl (no CAS) | 22.6 | 389 | — | — |
| Actin (0-500 cells) | 31.0-32.1 | 65-135 | 30.1-31.5 | 90-125 |

These results clearly demonstrate that q-MSP can successfully be applied to CTCs from patients with prostate cancer.

1. A method for analyzing a biological specimen for the presence of cells specific for an indication comprising the steps of:
a) enriching cells from the specimen;
b) isolating nucleic acid and/or protein from the cells; and
c) analyzing the nucleic acid and/or protein to determine the presence, expression level or status of a Biomarker specific for the indication.

2. The method according to claim 1 wherein the biological specimen is selected from urine, blood, serum, plasma, lymph, sputum, semen, saliva, tears, pleural fluid, pulmonary fluid, bronchial lavage, synovial fluid, peritoneal fluid, ascites, amniotic fluid, bone marrow, bone marrow aspirate, cerebrospinal fluid, tissue lysate or homogenate or a cell pellet.

3. The method according to claim 1 wherein the indication is cancer, risk assessment of inherited genetic pre-disposition, identification of tissue of origin of a cancer cell such as a CTC, identifying mutations in hereditary diseases, disease status (staging), prognosis, diagnosis, monitoring, response to treatment, choice of treatment (pharmacologic), infection (viral, bacterial, mycoplasmal, fungal), chemosensitivity, drug sensitivity, metastatic potential or identifying mutations in hereditary diseases.

4. The method according to claim 1 wherein the cells are enriched by antibody/magnetic separation, fluorescence activated cell sorting, (FACs), filtration or manually.

5. The method according to claim 4 wherein the manual enrichment is by prostate massage.

6. The method according to claim 1 wherein the nucleic acid is nuclear, mitochondrial (homeoplasmy, heteroplasmy), viral, bacterial, fungal or mycoplasmal.

7. The method according to claim 6 wherein the nucleic acid is DNA or RNA.

8. The method according to claim 1 wherein the analysis is DNA analysis.

9. The method according to claim 8 wherein the DNA analysis is related to methylation—de-methylation, karyotyping, ploidy (aneuploidy, polyploidy), DNA integrity (assessed through gels or spectrophotometry), translocations, mutations, gene fusions, activation—de-activation, single nucleotide polymorphisms (SNPs), copy number or whole genome amplification to detect genetic makeup.

10. The method according to claim 8 wherein the analysis is RNA analysis.

11. The method according to claim 10 wherein the RNA analysis is related to q-RT-PCR, miRNA or post-transcription modifications.

12. The method according to claim 8 wherein the analysis is protein analysis.

13. The method according to claim 12 wherein the protein analysis is related to antibody detection, post-translation modifications or turnover.

14. The method according to claim 13 wherein the proteins are cell surface markers.

15. The method according to claim 14 wherein the cell surface markers are epithelial, endothelial, viral or cell type.

16. The method according to claim 1 wherein the presence of the Biomarker is related to viral/bacterial infection, insult or antigen expression.

17. The method according to claim 16 wherein the antigen is used to separate cells.

18. The method according to claim 1 wherein the analysis is used to obtain a molecular profile of the enriched cells.

19. A method of determining metastatic potential of a cell from a biological specimen comprising the steps of:
a) enriching cells from the specimen;
b) isolating nucleic acid and/or protein from the cells; and
c) analyzing the nucleic acid and/or protein to determine the presence, expression level or status of a Biomarker specific for metastatic potential.

20. A method of identifying mutations in hereditary diseases from a cell from a biological specimen comprising the steps of:
a) enriching cells from the specimen;
b) isolating nucleic acid and/or protein from the cells; and
c) analyzing the nucleic acid and/or protein to determine the presence, expression level or status of a Biomarker specific for a hereditary disease.

21. A method of preserving genetic material from a cell from a biological specimen comprising the steps of:
a) enriching cells from the specimen;
b) isolating nucleic acid and/or protein from the cells; and
c) preserving the nucleic acid and/or protein.

22. A method of making a tumor cell vaccine comprising the steps of
a) obtaining a biological specimen
b) enriching cells from the specimen;
c) isolating nucleic acid and/or protein from the cells; and
d) using the nucleic acid and/or protein to formulate the vaccine.

23. A composition comprising the nucleic acid and/or protein obtained by the method of claim 1.

24. A composition comprising an oligonucleotide selected from SEQ ID NOs: 1-94.

24. A kit comprising biomarker detection agents for performing the method according to claim 1.

25. An article comprising biomarker detection agents for performing the method according to claim 1.


SEQUENCE LISTING (53 sequences)

SEQ ID NO: 1 - DNA, 18 bp, human
tcggggattttagggcgt

SEQ ID NO: 2 - DNA, 21 bp, human
acgaaaactacgacgacgaaa

SEQ ID NO: 3 - DNA, 24 bp, human
gatataaggttagggataggatag

SEQ ID NO: 4 - DNA, 22 bp, human
aaccaataaaacctactcctcc

SEQ ID NO: 5 - DNA, 47 bp, human
cgcacggcgaactcccgccgacgtgcgtgtagcggtcgtcggggttg

SEQ ID NO: 6 - DNA, 22 bp, human
gccccaatactaaatcacgacg

SEQ ID NO: 7 - DNA, 52 bp, human
ccgcgcatcaccaccccacacgcgcggggagtatataggttggggaagtttg

SEQ ID NO: 8 - DNA, 27 bp, human
aacacacaataacaaacacaaattcac

SEQ ID NO: 9 - DNA, 22 bp, human
aatggccaaagcactgctctta

SEQ ID NO: 10 - DNA, 21 bp, human
acttgctgtttttgctcatgt

SEQ ID NO: 11 - DNA, 30 bp, human
atcgaatcaaaaaacaagcatggcctcaca

SEQ ID NO: 12 - DNA, 22 bp, human
cacccttcagggtcttgagatt

SEQ ID NO: 13 - DNA, 20 bp, human
tccgtttctgccagtgtgtc

SEQ ID NO: 14 - DNA, 24 bp, human
acagctgagcatgaaagctgcctt

SEQ ID NO: 15 - DNA, 22 bp, human
ccacacacagcctactttccaa

SEQ ID NO: 16 - DNA, 21 bp, human
tacccacgcgaatcactctca

SEQ ID NO: 17 - DNA, 27 bp, human
aacggcaatgcggctgcaacggcggaa

SEQ ID NO: 18 - DNA, 21 bp, human
agttgctgatggtcctcatgc

SEQ ID NO: 19 - DNA, 24 bp, human
cacttgtggattgattgtcttgga

SEQ ID NO: 20 - DNA, 23 bp, human
ccctctcccagcactgctacgca

SEQ ID NO: 21 - DNA, 21 bp, human
gagtacgtgggcctgtctgca

SEQ ID NO: 22 - DNA, 21 bp, human
ttgcactccttgggggtgaca

SEQ ID NO: 23 - DNA, 25 bp, human
accagtgtgccgtgccagccaagga

SEQ ID NO: 24 - DNA, 20 bp, human
ctcctggttctctgcctgca

SEQ ID NO: 25 - DNA, 24 bp, human
gacgtactgacttgggaatgtcaa

SEQ ID NO: 26 - DNA, 30 bp, human
aagctcaggacaacactcggaagatcattt

SEQ ID NO: 27 - DNA, 22 bp, human
ctgaggagtacgtgggcctgtc

SEQ ID NO: 28 - DNA, 21 bp, human
ttgcactccttgggggtgaca

SEQ ID NO: 29 - DNA, 27 bp, human
caaaccagtgtgccgtgccagccaatt

SEQ ID NO: 30 - DNA, 21 bp, human
gcttggtggttaaaacttacc

SEQ ID NO: 31 - DNA, 20 bp, human
tgaacagttctgttggtgta

SEQ ID NO: 32 - DNA, 29 bp, human
ctgcctgcctatgtgacgacaatccggtt

SEQ ID NO: 33 - DNA, 21 bp, human
tgacactggcaaaacaatgca

SEQ ID NO: 34 - DNA, 21 bp, human
ggtccttttcaccagcaagct

SEQ ID NO: 35 - DNA, 34 bp, human
ctttgctttccttggtcaggcagtataatccatt

SEQ ID NO: 36 - DNA, 19 bp, human
aaaaacaagcatggcctac

SEQ ID NO: 37 - DNA, 21 bp, human
cagcaagttgagagcagtcct

SEQ ID NO: 38 - DNA, 31 bp, human
catgagcaaaaacagcaagtcgtgaaatttt

SEQ ID NO: 39 - DNA, 20 bp, human
cgcccacctggacatctgga

SEQ ID NO: 40 - DNA, 23 bp, human
cactggtcgaggcacagtagtga

SEQ ID NO: 41 - DNA, 27 bp, human
gtcagcggcctggatgaaagagcggtt

SEQ ID NO: 42 - DNA, 22 bp, human
aatggccaaagcactgctctta

SEQ ID NO: 43 - DNA, 21 bp, human
acttgctgtttttgctcatgt

SEQ ID NO: 44 - DNA, 32 bp, human
atcgaatcaaaaaacaagcatggcctcacatt

SEQ ID NO: 45 - DNA, 22 bp, human
cacccttcagggtcttgagatt

SEQ ID NO: 46 - DNA, 20 bp, human
tccgtttctgccagtgtgtc

SEQ ID NO: 47 - DNA, 26 bp, human
acagctgagcatgaaagctgcctttt

SEQ ID NO: 48 - DNA, 22 bp, human
ccacacacagcctactttccaa

SEQ ID NO: 49 - DNA, 21 bp, human
tacccacgcgaatcactctca

SEQ ID NO: 50 - DNA, 29 bp, human
aacggcaatgcggctgcaacggcggaatt

SEQ ID NO: 51 - DNA, 21 bp, human
agttgctgatggtcctcatgc

SEQ ID NO: 52 - DNA, 24 bp, human
cacttgtggattgattgtcttgga

SEQ ID NO: 53 - DNA, 25 bp, human
ccctctcccagcactgctacgcatt